(19) United States
(12) Patent Application Publication
(10) Pub. No.: US 2024/0218059 A1
(43) Pub. Date: Jul. 4, 2024

(54) TGF-Beta 1 INHIBITORS FOR PREVENTING AND TREATING SARS-COV-2

(71) Applicant: The Regents of the University of Colorado, A Body Corporate, Denver, CO (US)

(72) Inventor: James Maloney, Englewood, CO (US)

(21) Appl. No.: 17/907,094

(22) PCT Filed: Mar. 23, 2021
(86) PCT No.: PCT/US2021/023694

§ 371 Date: Sep. 22, 2022

Related U.S. Application Data
(60) Provisional application No. 62/993,696, filed on Mar. 23, 2020.

(51) Int. Cl. C07K 16/22; A61K 31/454; A61K 31/4709; A61K 39/00; A61P 31/12

(52) U.S. Cl. CPC C07K 16/22; A61K 31/454; A61K 31/4709; A61P 31/12; A61K 2039/505

(57) ABSTRACT

Prophylaxis and treatment method for SARS-CoV-2 infection are provided. Methods include administering a therapeutically effective amount of at least one TGF-β1 inhibitor to a subject at risk of contracting, or having a SARS-CoV-2 infection.

### CROSS-REFERENCE TO RELATED APPLICATION

This application claims the benefit of U.S. Provisional Application No. 62/993,696, entitled “TGF-B1 Inhibitors for Preventing and Treatment SARS-COV-2,” filed Mar. 23, 2020, the disclosure of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Mar. 22, 2021, is named 517709_36_SL.txt and is 7,836 bytes in size.

### BACKGROUND

Coronaviruses are a group of large, enveloped, positive-sense, single stranded RNA viruses. Originating in bats, zoonotic coronaviruses have been present in humans for at least 500-800 years, and are often the cause of the common cold. Of the four genera of coronaviruses (alpha, beta, gamma, and delta), characterized by different antigenic cross-reactivity and genetic makeup, only alpha- and betacoronavirus genera include strains pathogenic to humans.

Of the known coronavirus species, only six are known to cause disease in humans: HCoV-229E, HCoV-OC43, HCoV-NL63, HCoV-HKU1, severe acute respiratory syndrome coronavirus (SARS-COV) and Middle East respiratory virus coronavirus (MERS-COV). HCOV-229E, HCoV-OC43, HCoV-NL63, and HCoV-HKU1 are endemic locally, and have been associated mainly with mild, self-limiting disease, whereas SARS-COV and MERS-COV can cause severe illness. SARS-COV and MERS-COV are betacoronaviruses, and are among the pathogens included in the World Health Organization's list of high-priority threats.

Coronaviruses are so named for the crown-like spikes on their surface, and have two major envelope proteins. The S glycoprotein is a major antigen responsible for both receptor binding and cell fusion. The transmembrane glycoprotein (M) is involved in budding and envelope formation. A few coronavirus species have a third glycoprotein, the haemagglutinin-esterase (HE). The coronavirus genome is non-segmented, positive single-stranded RNA of about 26-32 kb, making it the longest RNA viral genome known, included from 7 to 10 open reading frames. Coronaviruses are capable of adapting quickly to new hosts through the processes of genetic recombination and mutation in vivo.

In late 2019, a new coronavirus began causing febrile respiratory illness in China. The virus, provisionally known as 2019-nCOV (2019 novel coronavirus; now known as SARS-COV-2), was first detected in Wuhan, China. SARS-COV-2 was sequenced and identified as a betacoronavirus belonging to the sarbecovirus, with 75-80% similarity in genetic sequence to SARS-COV. The animal host of SARS-COV-2 is presumed to be a bat, although an intermediate hose may also have been involved. Although the initial cases were a result of zoonotic transmission, human-to-human transmission was documented soon after, in both healthcare setting and familial clusters.

Following an incubation ranging from 2-14 days, SARC-CoV-2 infection manifests as a respiratory illness termed COVID-19 (coronavirus disease 2019), with symptoms including fever, cough, and dyspnea. An early description of 41 clinical cases described patients as having serious, sometimes fatal, pneumonia, with clinical presentations very similar to those of SARS-COV. Patients with the most severe illness developed acute respiratory distress syndrome (ARDS), requiring ICU admission and oxygen therapy.

According to the World Health Organization, as of Mar. 22, 2020, a total of 294, 110 confirmed cases of COVID-19 had been reported worldwide, with cases being reported in 187 different countries, areas, or territories. Although the early case-fatality rate appeared to be low, the rapid spread and ease of transmission of the virus, even by asymptomatic individuals, has caused global alarm; if easily transmissible, a virus poses a significant risk at the population level. Indeed, the WHO declared SARS-COV-2 infection to be a pandemic on Mar. 11, 2020.

As of Mar. 22, 2020, several drugs under investigation for treating COVID-19, although SARS-COV-2 infection and the resulting disease largely remain untreatable. The virus is unique in that it has enhanced communicability relative to, for example, influenza, and an unusually mild and prolonged prodrome for a respiratory virus before it leads to critical illness (i.e., ARDS). Together, these factors suggest poor recognition by the human immune system, resulting in a higher mortality rate than influenza infection.

### SUMMARY

In a first example (“Example 1”), provided herein is a method of preventing or treating a SARS-COV-2 infection in a subject in need thereof comprising administering to the subject a therapeutically effective amount of at least one transforming growth factor beta 1 (TGF-β1) inhibitor.

In certain embodiments of Example 1, the at least one TGF-β1 inhibitor inhibits activation of TGF-β1 by SARS-COV-2 NSP15. In certain embodiments, the at least one TGF-β1 inhibitor blocks interaction of TGF-β1 and SARS-COV-2 NSP15. In certain embodiments, the at least one TGF-β1 inhibitor blocks interaction of TGF-β1 and the KRFK domain of SARS-COV-2 NSP15. In certain embodiments, the at least one TGF-β1 inhibitor binds to KRFK (SEQ ID NO: 2) of SARS-COV-2 NSP15. In certain embodiments, the at least one TGF-β1 inhibitor binds to LLIGLAKRFKESPFEL (SEQ ID NO: 4) of SARS-COV-2 NSP15.

In a second example (“Example 2”), medicaments for preventing or treating a SARS-COV-2 infection in a subject in need thereof, comprising at least one transforming growth factor beta 1 (TGF-β1) inhibitors and one or more pharmaceutically acceptable carriers, vehicles, and/or excipients.

In another example (“Example 3”), further to Example 1 or Example 2, the at least one TGF-β1 inhibitor is selected from Galunisertib (LY2157299), LY580276, LY550410, SB505124, GS-1423, AVID200, Fresolimumab, LY2382770, LY3022859, and XOMA089.

In another example (“Example 4”), further to any one of Examples 1-3, the at least one TGF-β1 inhibitor is or includes a selective TGF-β1 inhibitor.

In another example (“Example 5”), further to any one of Examples 1-4, the at least one TGF-β1 inhibitor is targetable to lungs of the subject.

In another example (“Example 6”), further to any one of Examples 1-5, the subject is at risk of infection by SARS-COV-2.

In another example (“Example 7”), further to any one of Examples 1-5, the subject is infected by SARS-COV-2.

In another example (“Example 8”), further to any one of Examples 1-7, the subject is administered two or more doses of the at least one TGF-β1 inhibitors.

In another example (“Example 9”), further to Example 2, the medicament is formulated for delivery to the lungs of the subject.

### BRIEF DESCRIPTION OF THE DRAWINGS

The accompanying drawings are incorporated into and form a part of the specification to illustrate several examples of the present disclosure. The drawings simply illustrate examples of the disclosure and are not to be construed as limiting the disclosure to the illustrated and described examples. Further features and advantages will become apparent from the following, more detailed, description of the various aspects, embodiments, and configurations of the disclosure, as illustrated by the drawings referenced below.

FIG. 1A presents the amino acid sequence of SARS-COV-2 NSP15 (SEQ ID NO: 1). The KRFK motif is in bold.

FIG. 1B presents an alignment of viral sequences in the KRFK region of SARS-COV-2 NSP15. The alignment performed using Clustal alignment package within DS Gene software.

FIG. 2 is a photograph depicting TGF-β1 mRNA localization in human lung. FIG. 2A—bronchiolar epithelial cells. FIG. 2B—alveolar macrophage. FIG. 2C—pulmonary endothelial cells. FIG. 2D—mesenchymal cells. Original magnification: FIG. 2A=200×; FIGS. 2B, 2C, 2D=1000×. Internal scale bar=50 μm. Prior art (Eur Respir J, 1996, 9, 2501-2507).

FIG. 3A is a photograph presenting the results of an RNAse protection assay, indicating TSP1 expression in mouse lung. Prior art (Adapted from Lawler et al., J Clin Invest, 1998, 101 (5), 982-992)

FIG. 3B is a photograph depicting TSP1 protein localization in human lung at the epithelial basement membrane. Prior art (American Journal of Pathology, 1995, 147(6), 1759-69).

FIG. 4A is a schematic illustrating possible mutational events for a common cold coronavirus occurring in animal hosts, leading to increased infectivity and replication in SARS-COV-2.

FIG. 4B is a schematic illustrating a strategy for prophylaxis and treatment of SARS-COV-2 infection.

FIG. 5 is a graph of in vitro activation data for human latent TGF-β1 contacted by specific peptides, using a TGF-β1 responsive mink lung cell line (MLEC). SARSCOV2: 16 amino acid synthetic peptide of KRFK region of SARS-CoV2 NSP15 protein. TSP1: 16 amino acid peptide of KRFK region of human thrombospondin-1 (positive control). SCR: computer-scrambled peptide with no KRFK sequence. RLU: relative light unit. Significance was tested by two-tailed paired t-tests on an N of at least 6 experiments.

FIG. 6 is a graph of activation data of human latent TGF-β1 contacted by recombinant full-length hexameric SARS-COV-2 NSP15 protein (SARSCOV2 rNSP1), using a TGF-β1 responsive mink lung cell line (MLEC) (right panel). Positive control data is shown in the left panel. TSP: thrombospondin-1 protein. RLU: relative light unit. Significance was tested by two-tailed paired t-tests on an N of at least 6 experiments. Data are graphed as mean+/−SD.

FIG. 7 is a graph of data showing the inhibitory activity of representative TGF-β1 receptor inhibitors (SB505124 and galunisertib) that bind to and inhibit TGF-β receptor 2 (TGFRβ2) activity and a green tea catechin (ECGC; inhibits kinase function of TGFRβ2) on active TGF-β1 using a TGF-β1 responsive mink lung cell line (MLEC). RLU: relative light unit. Significance was tested by two-tailed paired t-tests on an N of at least 6 experiments. Data are graphed as mean+/−SD.

FIG. 8 is a graph of data showing the inhibitory activity of SB505124, galunisertib, and an anti-TGFβ1 antibody on TGFβ1 signaling after activation of latent TGF-β1 by NSP15, using a TGF-β1 responsive mink lung cell line (MLEC). Human latent TGF-β1 was activated by different concentrations of SARS-COV-2 NSP15 recombinant protein (rNSP15) before addition to cells. Significance was tested by two-tailed paired t-tests on an N of at least 6 experiments. Data are graphed as mean+/−SD.

### DETAILED DESCRIPTION

In the following sections, various compositions and methods are described in order to detail various embodiments. Practicing the various embodiments does not require the employment of all of the specific details outlined herein, but rather concentrations, times, and other specific details may be modified. In some cases, well known methods or components have not been included in the description.

### Definitions

As used herein, “treat” in reference to an infection or condition means: (1) to ameliorate or prevent the infection condition or one or more of the biological manifestations of the infection or condition, (2) to interfere with (a) one or more points in the biological cascade that leads to or is responsible for the condition or (b) one or more of the biological manifestations of the infection or condition, (3) to alleviate one or more of the symptoms or effects associated with the infection or condition, and/or (4) to slow the progression of the infection or condition, or one or more of the biological manifestations of the condition. The terms “prevent,” “preventing,” and the like are to be understood to refer to a method of blocking the onset of infection and/or its attendant disease or symptoms. “Prevent” also encompasses delaying or otherwise impeding the onset of an infection and/or its attendant disease or symptoms, as well as minimizing/reducing infection.

As used herein, “therapeutically effective amount” in reference to an agent means an amount of the agent sufficient to prevent or treat the subject's infection or condition but low enough to avoid serious side effects at a reasonable benefit/risk ratio within the scope of sound medical judgment. The safe and effective amount of an agent will vary with the particular agent chosen (e.g. consider the potency, efficacy, and half-life of the compound); the route of administration chosen; the infection or condition being treated; the severity of the infection or condition being treated; the age, size, weight, and physical condition of the patient being treated; the medical history of the patient to be treated; the duration of the treatment; the nature of concurrent therapy; the desired therapeutic effect; and like factors, but can nevertheless be determined by the skilled artisan.

For any compound, agent, or composition, the therapeutically effective amount can be estimated initially either in cell culture assays or in animal models, usually rats, mice, rabbits, dogs, or pigs. The animal model may also be used to determine the appropriate concentration range and route of administration. Such information can then be used to determine useful doses and routes for administration in humans. Potential routes for administration include intravenous injection, subcutaneous injection, intramuscular injection, oral administration, intranasal administration, and inhalation (by nebulizer). Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

A “subject” means any individual having, having symptoms of, or at risk of infection by SARS-COV-2 and/or its associated disease, COVID-19. Symptoms of SARS-COV-2 infection/COVID-19 include fever, dry cough, dyspnea, and fatigue or myalgia. SARS-COV-2 infection may result in acute respiratory distress syndrome (ARDS), with symptoms of shortness of breath, rapid breathing, dizziness, rapid heart rate, and excessive sweating. A subject may be human or non-human, and may include, for example, animals or species used as “model systems” for research purposes. In certain embodiments, the subject is a human patient having or at risk of infection by SARS-COV-2 and/or its associated disease, COVID-19.

As used herein, a “pharmaceutical composition” is a formulation containing a compound or agent (e.g., TGF-β1 inhibitors) in a form suitable for administration to a subject. Compounds and agents disclosed herein each can be formulated individually or in any combination into one or more pharmaceutical compositions. Accordingly, one or more administration routes can be properly elected based on the dosage form of each pharmaceutical composition. Alternatively, a compound or agent disclosed herein and one or more other therapeutic agents described herein can be formulated as one pharmaceutical composition.

As used herein, “inhibitor” refers to a small molecule, antibody, antisense oligo, peptide, siRNA, or the like, that i) binds to a protein such as an enzyme and decrease its activity, or ii) binds to a receptor—but does not activate it—thereby blocking the action of the receptor's ligand, or iii) binds to the ligand itself, blocking it from interacting with its receptor. An inhibitor that binds to a receptor but does not activate it may also be referred to as an antagonist. An antagonist may be a competitive antagonist (binds to the same site on a receptor as the agonist but does not activate the receptor thereby blocking the action of the agonist) or a non-competitive antagonist (binds to a non-agonist and prevents the action of an agonist without affecting the agonist binding to the receptor).

### DETAILED DESCRIPTION

There are few effective treatments for COVID-19. Notably, SARS-COV-2 displays early immune evasion, then kills via the acute respiratory failure syndrome (ARDS). Further elucidation of immune mechanisms exploiting by SARS-COV-2 with identification of drug targets is critical.

The inventor has discovered that all SARS-COV-2 strains have a KRFK mutation in their NSP15 protein, and, as described herein, the inventor has recognized the significance of this finding and how this finding can explain key features of SARS-COV-2. Specifically, the protein TGF-beta 1 (TGF-β1) has a prominent role in ARDS development and immune evasion by microbes. Moreover, once freed from its latent state, TGF-β1 is a potent immune suppressant. TGF-β1 is stored in an inactive “latent” locked form that can only be opened by certain molecular keys. One of these keys is the rare “KRFK” domain (lysine-arginine-phenylalanine-lysine; SEQ ID NO:2) protein motif.

The inventor has tested the SARS-COV-2 NSP15 KRFK region using regional peptides. The data demonstrate that the SARS-COV-2 NSP15 KRFK region activates latent TGF-β1 (whereas the same amino acids, in a peptide of scrambled sequence without a KRFK domain, do not activate latent TGF-β1), and shows potent activation with recombinant SARS-COV-2 NSP15 protein. The inventor has further shown that commercially available TGF-β1 inhibitors are effective in blocking TGF-β1 effects on a lung epithelial cell line model relevant to COVID-19 illness, and relevant to the TGF-β1 released by the SARS-COV-2 NSP15 protein. Injurious effects from activation of TGF-β1 by the SARS-COV-2 NSP15 protein can be antagonized by TGF-β1 inhibitors, including inhibitors targeted to the latent TGF-β1 binding site of SARS-COV-2 NSP15. Such therapies can improve prevention and treatment of human SARS-COV-2 related illness.

Accordingly, embodiments of the present disclosure provide curative and prophylactic therapies for SARS-COV-2 infection and/or associate dissociate disease, COVID-19. Methods for preventing or treating SARS-COV-2 in a subject are provided, the methods including administering to the subject a therapeutically effective amount of a TGF-β1 inhibitor.

Other embodiments of the present disclosure provide prophylactic therapies capable of preventing, including slowing, a SARS-COV-2 infection in a subject. Methods for preventing a SARS-COV-2 infection in a subject include administering to a subject at risk of SARS-COV-2 infection a therapeutically effective amount of a TGF-β1 inhibitor.

In certain embodiments, the TGF-β1 inhibitor is one or more of Galunisertib (LY2157299), LY580276, LY550410, SB505124, GS-1423, AVID200, Fresolimumab, LY2382770, LY3022859, and XOMA089. In particular embodiments, the TGF-β1 inhibitor is a selective TGF-β1 inhibitor (e.g., LY2382770).

One or more additional compounds affecting (e.g., treating or preventing) SARS-COV-2 may be administered to the subject in addition to the TGF-β1 inhibitor. In some embodiments, methods described herein further include administering to the subject a therapeutically effective amount of one or more of remdesivir, chloroquine and/or hydroxychloroquine, combination drug ritonavir/lopinavir, ritonavir/lopinavir and interferon-beta, and OT-101 (trabedersen). Other compounds affecting or modulating SARS-COV-2 infection and its associated condition are also contemplated.

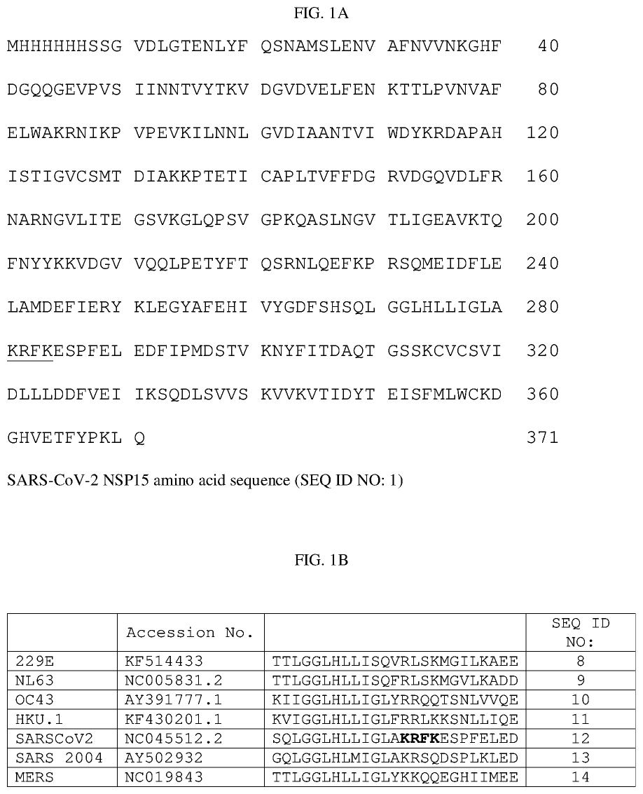

As illustrated by FIG. 1A and described here for the first time, a KRFK domain appears in the C-terminus of nonstructural protein 15 (NSP15) of severe acute respiratory syndrome coronavirus 2 (SARS-COV-2). As described herein, the gain of a KRFK domain indicates a role for transforming growth factor beta 1 (TGF-β1) in the escape of detection of SARS-COV-2 by the immune system, and the ability to prevent or treat SARS-COV-2 infection by inhibiting TGF-β1 signaling. An uncommon domain, the KRFK (SEQ ID NO: 2) peptide domain is the canonical domain contained by proteins having the ability to activate TGF-β1.

All ten SARS-COV-2 genomes analyzed included the KRFK domain, and included viruses isolated in China, Africa, Japan, the United States, and Italy, amongst others.

The KRFK domain is extremely rare in viruses. See, e.g., FIG. 1B. A 2014 coronavirus strain appeared to have the domain, but in the context of otherwise benign, common cold features due to a lack of other permissive mutations. A bioinformatics search of the SARS 2004, Ebola, 1918 Influenza, 2009 H1N1 Influenza, MERS, and 3 common cold coronaviruses by the inventor returned no evidence of KRFK domains in these viruses. The acquisition of this domain goes far to explain the unique features of SARS-COV-2—unchecked early viral proliferation with absence of the usual and early fever, headache, and myalgias otherwise typical for an early host immune response, a high rate of person-to-person transmission during early infection, and late progression to acute respiratory distress syndrome (ARDS) and related death when unopposed viral proliferation in lung epithelial cells becomes overwhelming. This virus “cloaks” itself from immune recognition, using TGF-β1, and thus amplifying the effect of other proposed pathogenic features (such as a predicted increase in spike protein recognition of the ACE2 receptor on lung epithelial cells, enhancing viral entry). This SARS-Cov-2 KRFK mutation appears to be the last in a “perfect storm” of genetic events that led to the severe pathogenicity of SARS-COV-2 and the COVID-19 pandemic—but importantly the KRFK mutation can be directly targeted by existing therapeutics

A pluripotent cytokine, TGF-β1 is one of three isoforms of TGF-β, playing in an important role in controlling the immune system. Most evidence in the literature indicates TGF-β1 to be involved in immune suppression, with TGF-β2 seemingly playing a minor role in immune suppression. TGF-β1 has been demonstrated to have an essential role in establishing immunological homeostasis and tolerance by way of immune suppression, inhibiting the expansion and proliferation of many components of the immune system, including T cells. Synthesized as a precursor molecule containing a propeptide in addition a TGF-β1 homodimer, the TGF-β1 homodimer interacts with Latency Associated Peptide (LAP) to form the intracellular Small Latent Complex (SLC). The SLC is bound by Latent TGF-β-Binding Protein (LTBP) to form the Large Latent Complex (LLC), which is secreted from the cell. Following secretion, further processing is required in order to release active TGF-β1. As different cellular mechanisms require distinct TGF-β1 levels for signaling, the latent complex provides an opportunity for controlling TGF-β1 signaling.

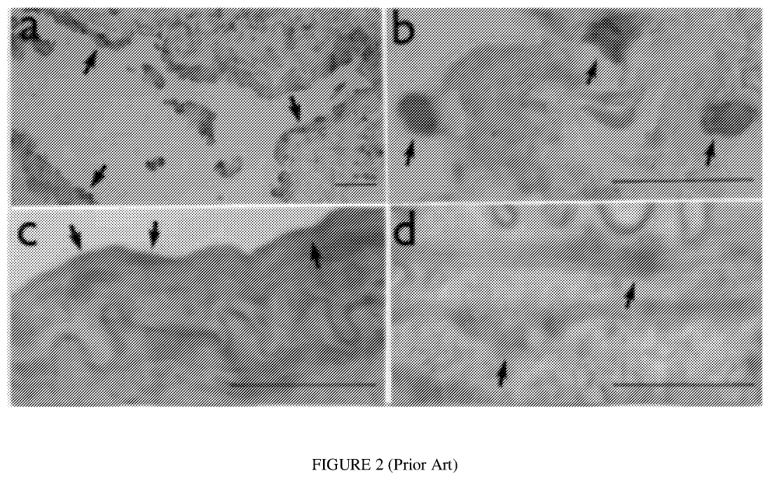

As depicted by FIG. 2, TGF-β1 is highly expressed in human lung in regions where coronaviruses have been demonstrated to bind.

Several parasites, including the malaria parasite, schistosomes, and Leishmania spp., have evolved to upregulate host TGF-β1 in order to avoid immune detection in humans or other animals, allowing the parasites to proliferate and remain undetected until they cause severe disease.

Thrombospondin-1 (TSP1) is a widely expressed matricellular protein, and is a major endogenous activator of TGF-β1, thus playing a significant role in immune suppression and tolerance. TSP1 includes a KRFK domain, which is critical for latent TGF-β1 activation. The KRFK domain binds to a conserved sequence in the LAP, which disrupts LAP-mature domain interactions to expose the TGF-β1 receptor binding sequences, thereby activating TGF-β1. TSP2, which lacks the KRFK domain, does not activate TGF-β1, and can act as a competitive antagonist of TSP1-mediated TGF-β1 activation. Further, a KRFK peptide alone has been shown to be sufficient to activate latent TGF-β1.

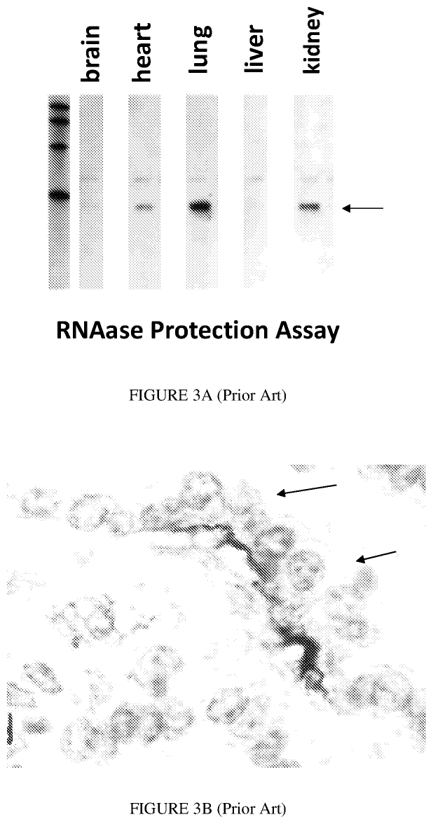

As depicted in FIG. 3B, TSP1 is highly expressed in human lung, with highest concentrations at the lung epithelial basement membrane, where coronaviruses bind. This indicates that TGF-β1 activation normally occurs at the location where coronaviruses bind in the lung, demonstrating that a coronavirus having a KRFK domain would be appropriately localized to activate TGF-β1.

As with TGF-β1-upregulating parasites, SARS-COV-2 has developed a mechanism allowing the upregulation of TGF-β1. The KRFK domain of SARS-COV-2 NSP15 identified by the inventors provides evidence that the virus can evade immune detection by activating TGF-β1. Indeed, the subjects infected with SARS-COV-2 may be asymptomatic for about 2 days to about 14 days before symptoms begin to appear. Further SARS-COV-2 has an unusually mild and prolonged prodrome before it leads to critical illness (ARDS), indicating that the virus is evading immune detection and/or minimizing immune response.

While enzymes such as mammalian furin, mammalian plasmin, and influenza neuraminidase are capable of activating latent TGF-β1, they do so more slowly and require micromolar concentrations (like most enzymes). The KRFK domain (of TSP1) releases latent TGF-Beta rapidly and at a 1,000-fold higher potency, requiring only nanomolar concentrations.

Interestingly, in addition to a KRFK domain, SARS-COV-2 NSP15 also includes several ancillary WXXW (SEQ ID NO:3) peptide domains, which can aid in anchoring NSP15 to the intercellular matrix, where a large store of latent TGF-β1 resides (see FIG. 2).

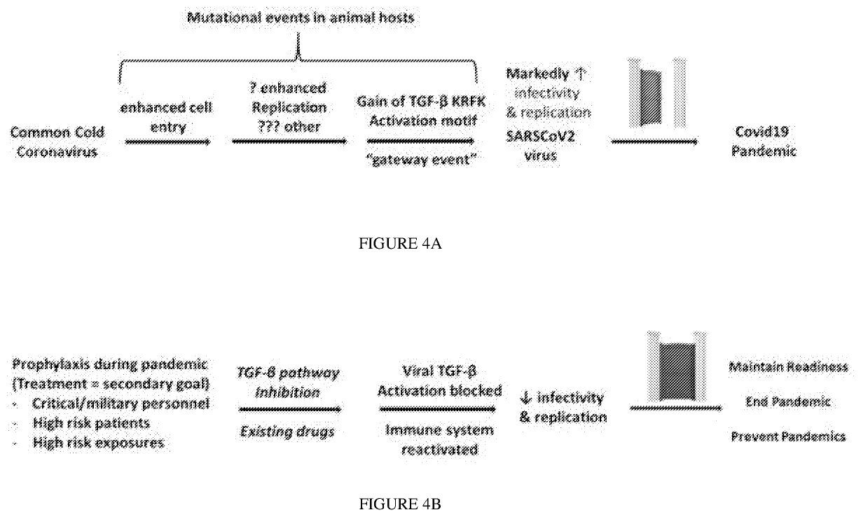

As depicted in FIG. 4A, the gain of a TGF-β1 KRFK activation motif appears to have acted as a “gateway event” in the evolution of SARS-COV-2. In combination with other gain-of-pathogenicity mutations in coronavirus (such as enhanced ability to enter lung epithelial cells via ACE2 receptor via an altered viral spike protein), the inclusion of a KRFK domain in NSP15—a protein known to mediate coronavirus evasion of dsRNA sensors, as well as being linked with low-level human immune system suppression in, for example, the common cold—has provided for the creation of a pandemic virus. The potent KRFK domain provides a selective advantage by releasing tissue stores of otherwise latent (i.e., inactive) immunosuppressive TGF-β1 wherever virus replicates, allowing the virus to ‘cloak’ itself from local immune attack. This allows the virus to proliferate with minimal host symptoms during early infection, leading to extensive lung replication before the immune system is able to mount an initial response (or even a fever) and likely leading to the high mortality from acute respiratory distress syndrome (ARDS) seen during the 2019-2020 pandemic. Over days, billions of copies of the viral NSP15 KRFK motif are produced, and trigger a vicious cycle of TGF-β1 activation, immune suppression, and further viral replication. Accordingly, prophylaxis and/or treatment of SARS-COV-2 infection may be achieved by inhibiting the TGF-β1 pathway with an inhibitor of the KRFK domain of (SEQ ID NO: 2) of SARS-COV-2 NSP15.

As depicted in FIG. 4B, prophylaxis and/or treatment of SARS-COV-2 infection may be achieved by inhibiting the TGF-β1 pathway using existing drugs. This can ‘uncloak’ the virus, allowing for earlier immune detection (i.e., prophylaxis) or reactivation of the immune system (i.e., treatment), thereby decreasing SARS-COV-2 infectivity and replication.

Describe herein for the first time is the use of TGF-β1 inhibitors for the prophylactic prevention or treatment of SARS-COV-2 infection. Methods provided herein include administering to a subject a therapeutically effective amount of at least one TGF-β1 inhibitor. Useful TGF-β1 inhibitors may prevent TGF-β1 activation, or may interfere with TGF-β1 binding to its receptor. Augmentation of early anti-viral host defenses will minimize infection rates, length of illness, and infection severity for those infected, minimize virus shedding and its collateral effects within a population, and allow an earlier return to normal activities.

In some embodiments, at least one of the one or more TGF-β1 inhibitors is a selective TGF-β1 inhibitor. As used herein, “selective TGF-β1 inhibitor” refers to an inhibitor that preferentially inhibits TGF-β1 over TGF-β2 and TGF-β3.

In certain embodiments, the at least one TGF-β1 inhibitor inhibits activation of TGF-β1 by SARS-COV-2 NSP15.

In certain embodiments, the at least one TGF-β1 inhibitor blocks interaction of TGF-β1 and SARS-COV-2 NSP15.

In certain embodiments, the at least one TGF-β1 inhibitor blocks interaction of TGF-β1 and the KRFK domain of SARS-COV-2 NSP15.

In certain embodiments, the at least one TGF-β1 inhibitor binds to KRFK (SEQ ID NO: 2) of SARS-COV-2 NSP15.

In certain embodiments, the at least one TGF-β1 inhibitor binds to LLIGLAKRFKESPFEL (SEQ ID NO: 4) of SARS-COV-2 NSP15.

In certain embodiments, the at least one TGF-β1 is selected from Galunisertib (LY2157299), LY580276, LY550410, SB505124, GS-1423, AVID200, Fresolimumab, LY2382770, LY3022859, and XOMA089.

Developed by Eli Lilly, galunisertib (LY2157299), LY580276, and LY550410 are small molecule TGF-β signaling inhibitors that are being investigated for anti-tumor activity. Galunisertib, LY580276, and LY550410 treatments block signaling through the heteromeric TGFβ receptor complex. Galunisertib may be administered, for example, orally at 75 mg/kg/day.

Developed by GlaxoSmithKline SB505124 is a small molecule inhibitor of the TGF-β type I receptor, blocking signaling through the receptor.

GS-1423 is an anti-CD73-TGFβ-Trap bifunctional antibody being investigated by Gilead Sciences for use in treating advanced solid tumors. GS-1423 may be administered, for example, intravenously at doses of up to about 30 mg/kg once approximately bi-weekly.

AVID200 is a TGFβ trap with antibody-like properties, which is selective inhibitor of TGF-β1 and 3, sparing TGF-β2. AVID200 may be administered, for example, intravenously at a dose of about 180 mg/m², about 550 mg/m², or about 1,100 mg/m².

Fresolimumab is an anti-TGF-β monoclonal antibody that neutralizes all isoforms of TGF-β, and is being investigate for use in treatment of several cancers, including malignant melanoma, renal cell carcinoma, and metastatic breast cancer. Fresolimumab can be administered, for example, intravenously at does up to about 15 mg/kg about every 4 weeks.

LY2382770 is a recombinant humanized monoclonal antibody directed to TGF-β, and is specific for TGF-β1. LY2382770 was being investigated for use in diabetic nephropathy. LY2382770 can be administered, for example, subcutaneously at a dose of about 2 mg, about 10 mg, about 20 mg, about 30 mg, about 40 mg, or about 50 mg.

LY3022859 is an anti-TGF-β receptor type II monoclonal antibody that inhibits receptor-mediated signaling activation. LY3022859 can be administered, for example, intravenously about every 2 weeks at a dose of about 1.25 mg/kg, about 12.5 mg/kg, or about 25 mg/kg.

XOMA089 is an anti-TGF-β1 antibody that specifically neutralizes TGF-β1 and TGF-β2 ligands.

In certain embodiments, the at least one TGF-β1 inhibitor is targetable to the lungs of the subject. For example, the at least one TGF-β1 inhibitor is formulated for delivery into the respiratory tract via a nebulizer.

As provided herein, a TGF-β1 inhibitor can be administered prophylactically or as a treatment for SARS-COV-2 infection. In some embodiments, at least one TGF-β1 inhibitor is administered to a subject at risk of infection by SARS-COV-2. Such subjects include, for example, first responders; doctors, nurses, and other medical professionals and staff; military personnel; and close friends and family of subjects infected by SARS-COV-2. In other embodiments, at least one TGF-β1 inhibitor is administered to a subject infected by SARS-COV-2.

In certain embodiments, a prophylactic or therapeutic effect may require administration of two or more doses of the at least one TGF-β1 inhibitor.

Also provided are medicaments for preventing or treating a SARS-COV-2 infection in a subject, the medicaments including at least one TGF-β1 inhibitor described herein.

Also provided is a peptide comprising the sequence KRFK (SEQ ID NO: 2), a peptide comprising the sequence LLIGLAKRFKESPFEL (SEQ ID NO: 4) or a fragment of the same comprising KRFK and having 5, 6, 7, 8, 9, 10, 11, 12, 13, 14, or 15 amino acids of SEQ ID NO: 4, such as a fragment comprising amino acids 6-10, or amino acids 5-10, or amino acids 7-11, and so forth, of SEQ ID NO: 4, or a peptide sequence at least 75%, at least 80%, at least 85%, or at least 90% identical to SEQ ID NO: 4), or a nucleic acid encoding the same. The peptide or nucleic acid can be in a pharmaceutical composition that comprises one or more pharmaceutically acceptable adjuvants, carriers, vehicles, and/or excipients.

Carriers, vehicles, excipients, adjuvants, diluents and auxiliary substances are known in the art and may be present in the pharmaceutical compositions. An adjuvant refers to an agent that non-specifically enhances the immune response to an antigen. Exemplary adjuvants are known in the art, and include inorganic compounds such as alum and aluminum salts, oils such as mineral oil, bacterial products such as inactivated mycobacteria or bacterial lipopolysaccharides, plant saponins, cytokines, squalene, Vitamin E, glucans, dextrans, and the like, and combinations thereof, such as Freund's adjuvant (complete or incomplete). An adjuvant may be selected to be a preferential inducer of either a TH1 or a TH2 type of response. The composition optionally further comprises one or more additional antigens. Examples of such additional antigens are other SARS-COV-2 proteins and/or capsular polysaccharides.

In certain embodiments, the peptide (or nucleic acid encoding the same) or pharmaceutical composition comprising the peptide (or nucleic acid) is used in a method of inducing an immune response to SARS-COV-2 in a subject. Accordingly, also provided is a method of inducing an immune response to SARS-COV-2 in a subject, the method comprising administering a therapeutically or prophylactically effective amount of a peptide comprising the sequence KRFK (SEQ ID NO: 2), a peptide comprising the sequence LLIGLAKRFKESPFEL (SEQ ID NO: 4) or a fragment of the same comprising KRFK and having 5, 6, 7, 8, 9, 10, 11, 12, 13, 14, or 15 amino acids of SEQ ID NO: 4, such as a fragment comprising amino acids 6-10, or amino acids 5-10, or amino acids 7-11, and so forth, of SEQ ID NO: 4, or a peptide sequence having at least 75%, at least 80%, at least 85%, or at least 90% identical to SEQ ID NO: 4), or a nucleic acid encoding the same, or a pharmaceutical composition comprising a peptide comprising the sequence KRFK (SEQ ID NO: 2), a peptide comprising the sequence LLIGLAKRFKESPFEL (SEQ ID NO: 4), or a peptide sequence having at least 75%, at least 80%, at least 85%, or at least 90% identical to SEQ ID NO: 4, or a nucleic acid encoding the same.

Also provided is use of the pharmaceutical composition of a peptide comprising the sequence KRFK (SEQ ID NO: 2) or a pharmaceutical composition comprising the same to induce an immune response in a subject in need thereof.

### Benefits Over Prior Art

In March 2020, Mateon Therapeutics announced plans to investigate its lead compound OT-101 (trabedersen; AP12009) as a treatment for COVID-19 (i.e., SARS-COV-2 infection). A TGF-β2-specific antisense oligo therapeutic, OT-101 is being investigated as an inhibitor of cellular binding and viral replication, and as a suppressor of viral induced pneumonia. Having a sequence of 5′-CGGCATGTCTATTTTGTA-3′ (SEQ ID NO: 4), the antisense oligo binds to TGF-β2 mRNA, causing inhibition of protein translation, decreasing TGF-β2 protein levels. Although OT-101 is commonly referred to simply as a TGF-β inhibitor, it's specificity for TGF-β2 is critical. As provided by Mateon in its Apr. 30, 2019 United States Securities and Exchange Commission (SEC) filing, “therapeutics targeting TGF-β have not been successful and many have failed due to toxicity issues possibly due to inhibition of TGF-β1 essential functions. The high level of homology between the various TGF-β isoforms is making it impossible to create mAb or small molecule inhibitor without TGF-β1 cross-inhibition. Therefore, Oncotelic [Mateon] chose to target TGF-β2 only using OT-101 antisense approach. The sequence of OT-101 can only target TGF-β2 and does not have any impact on other TGF-β isotypes.”

It is well known that the three TGF-β isoforms have distinct effects. Indeed, null mice for the three isoforms have differing phenotypes (see, e.g., Sanford et al. (1997), Development 124:2659-2670). As provided herein, and unlike with OT-101, it is desirable for the prophylaxis and treatment of SARS-COV-2 to inhibit TGF-β1 signaling. In a setting of overwhelming TGF-β1 activation, as predicted herein is occurring in COVID-19, transient TGF-β1 inhibition is worthwhile, with its beneficial effects (i.e., uncloaking SARS-COV-2 to the immune system) outweighing any collateral negative effects.

As disclosed herein, SARS-COV-2 NSP15 protein has acquired, by mutational gain of a KRFK domain, the unfortunate ability to activate human latent TGF-β1. Notably, as contemplated herein and demonstrated in Examples 1-4, this ability can be blocked successfully using TGF-β1 inhibitors of multiple classes. Fortunately, multiple TGF-β1 inhibitors have been developed by the pharmaceutical industry and have undergone clinical trials for various cancers and inflammatory diseases. While none of these agents is yet FDA-approved, they are all eligible for repurposing to prevent SARS-Cov-2 NSP15 protein mediated activation of latent TGF-β1 and downstream effects of active TGF-β1 on cell injury and immune dysregulation.

Moreover, as contemplated herein, an extension of these data is the targeting the structural area of this KRFK domain by passive immunity using exogenous administration of engineered antibodies (or other types of inhibitors) to sterically inhibit latent TGF-β1 activation, or using this KRFK region in SARS-COV-2 vaccines to generate specific adaptive immunity and endogenous antibodies to sterically block this region and prevent activation of the extensive stores of latent TGF-β1 in the lung and human respiratory tree.

### EXAMPLES

The materials, methods, and embodiments described herein are further defined in the following Examples. Certain embodiments are defined in the Examples herein. It should be understood that these Examples, while indicating certain embodiments, are given by way of illustration only. From the disclosure herein and these Examples, one skilled in the art can ascertain the essential characteristics of this invention, and without departing from the spirit and scope thereof, can make various changes and modifications of the invention to adapt it to various usages and conditions.

### Example 1

The following experiment was performed to study the in vitro activation of human latent TGF-β1 by three different peptides. The activation of latent TGF-β1 was assessed using a TGF-β1 responsive cell line with a luciferase reporter driven by smad transcription factor binding elements. This cell line is derived from lung epithelial cells of mink (MLEC).

The peptides tested were: (1) a 16 amino acid synthetic peptide centered on the SARS-CoV2 NSP15 KRFK region (LLIGLAKRFKESPFEL; SEQ ID NO: 4); (2) a positive control peptide from the first described human KRFK-containing peptide thrombospondin-1 (TSP1) (DKRFKQDGGWSHWSPW; SEQ ID NO: 5); and (3) a computer-scrambled peptide (SCR) with no KRFK sequence (KFILALRKLLFESEGP; SEQ ID NO: 6). Peptides were designed by the inventor, then purchased commercially (Lifetine, Inc.).

Peptides were reconstituted in sterile water and added in the indicated micromolar (μM) concentrations to a reaction mix of 2 nM human latent TGF-β1 (Acrobio, Inc) in a 200 uL PBS reaction. The resulting reaction mix was incubated for 45 minutes at 37° C. in a water bath. At the end of the incubation, 80 uL of reaction mix was added to 320 uL of complete media (final concentration of peptide is InM), mixed, and divided in 100 uL aliquots among triplicate wells of mink lung epithelial cells (MLEC) per condition. MLEC were grown in 5% CO2 and 21% 02 in humidified incubators in passages 4-6 in complete media comprised of 10 percent FBS, high glucose DMEM, 1× penicillin-streptomycin, essential amino acids, and L-glutamine. When confluent, cells were passed by trypsinization and seeded at 20,000 cells/well of 96-well opaque optical plates with transparent bottoms, and incubated overnight at standard conditions. After 18 hours, the cells were washed once with PBS, then lysed with 30 uL of 1× Promega lysis buffer per well while agitating for 20 minutes. Luciferase signals were quantified in relative light units (RLU) after luciferase activity detection methods as described in Example 2.

As noted above, the activation of latent TGF-β1 was assessed using a TGF-β1 responsive cell line derived from lung epithelial cells of mink (MLEC). Mink are also is susceptible to SARS-COV-2 infection with high mortality because mink, like humans, express the ace-2 enzyme that is a receptor for the SARS-COV-2 spike protein binding that catalyzes viral entry into the cell. Thus mink lung cells serve as a useful model of this often fatal lung infection.

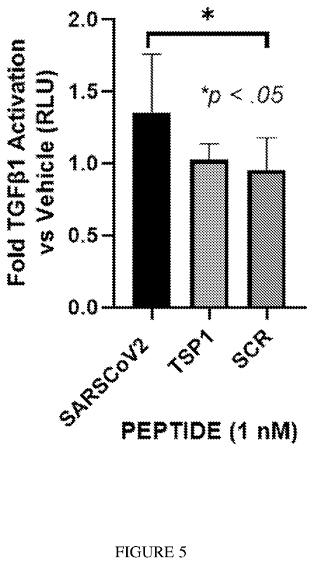

The data are shown in FIG. 5. Even isolated from its parent 250 kDa hexameric NSP15 protein, the short NSP15 KRFK-region peptide (SEQ ID NO″ 4) activates latent TGF-β1, but not when the KRFK sequence is removed by computerized scrambling of amino acids (SCR). Of note, in these experiments the positive control peptide, derived from human TSP1, was not a significant activator of latent TGF-β1.

The data from this experiment confirm that a peptide comprising the KRFK region from SARS-CoV-2 NSP15 activates latent TGF-β1.

### Example 2

The following experiment was performed to study the in vitro activation of human latent TGF-β1 by recombinant, full length hexameric SARS-COV-2 NSP15 protein.

NSP15 was produced in the inventor's laboratory using an artificial gene designed by the inventor and produced commercially in a pET151/D-TOPO plasmid (Thermo-Fisher) encoding the full-length monomeric complementary DNA sequence from a March 2020 SARS-COV-2 isolate reported to the NCBI, adding an N-terminal histidine-6 tag for purification, and codon-optimization for E. coli expression. After plasmid transduction and IPTG-stimulated expression of the transgene in three 3 liter flasks of LB broth with agitation in an incubator, media was centrifuged at 15,000×g and pellets were lysed with a Promega kit buffer and 4 pulses of 30 seconds of sonication. Cell lysates were centrifuged at 14,000×g and supernatants were combined and applied overnight in pbs ph 7.4 20 mm imidazole to a nickel bead column. Bound NSP15 was washed with 4 column volumes of this pbs imidazole buffer until the OD reading was zero, then eluted off with a 150-300 mM NaCl salt gradient in 500 mM imidazole using a fraction collector. Protein-rich fractions were pooled and buffer was exchanged using spin columns to PBS pH 7.4 and then applied to a MonoQ ion-exchange column. Bound NSP15 protein was eluted off using a salt gradient up to IM NaCl, then protein-rich fractions were pooled, buffer exchanged into 50 mM Tris, 5 mM manganese chloride and protein concentration was determined by Bradford assay. Fractions were stored in 50 percent glycerol and flash frozen before storage at −70° C. before use in experiments. Purity was confirmed by visualization of 42 kDa monomer bands on a reducing 6-10% gradient polyacrylamide gel followed by transfer to a membrane and western blotting using anti-His tag monoclonal antibody.

The ability of recombinant SARS-COV-2 NSP15 protein to activate human latent TGF-β1 was assessed by employing the TGF-β1 responsive MLEC cell line by methods described in Example 1. In these experiments 2 nM of latent human TGF-β1 in PBS pH 7.4 was mixed with 0-2.4 nM recombinant NSP15 or the positive control 10 nM recombinant human TSP1 protein for 45 minutes in a 200 μl volume in a 37° ° C. water bath. After 45 minutes 80 uL of the reaction mixture was added to 320 μL of complete media and divided into triplicate wells of confluent MLEC seeded the night before at 20,000 cells/well in opaque white 96-well optical plates, and incubated overnight at standard conditions. After 18 hours, the cells were washed once with PBS, then lysed with 30 μL of 1× Promega lysis buffer per well while agitating for 20 minutes. Luciferase signals were quantified in relative light units (RLU) as flash readings after the addition of 100 μL of Promega luciferase assay reagent per well, using a Tecan plate reader.

Each concentration (0.24 nM. 1.2 nM. 2.4 nM and 10 nM recombinant SARS-COV-2 NSP15 protein) was assayed in triplicate wells over 6-8 experiments. Triplicates were averaged and means compared by paired two-tailed t-tests using Graphpad software.

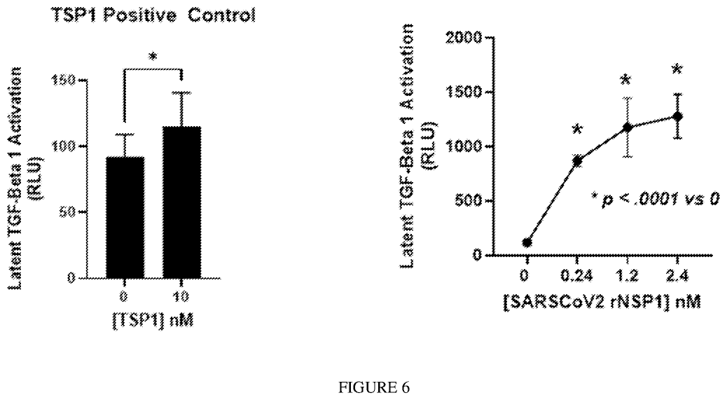

The data are shown in FIG. 6. NSP15 activation of latent TGF-β1 is shown in the right hand panel, and thrombospondin-1 activation of latent TGF-β1 is shown in the left hand panel. At 10 nM of NSP15, marked cell toxicity was observed, thus the data for this dose is not shown).

NSP15 activation of latent TGF-β1 is striking and occurs at nanomolar (nM) concentrations, is dose-responsive. At 10 nM of NSP15, marked cell toxicity was observed, thus the data for this dose is not shown. The NSP15 activation of latent TGF-β1 is more potent than that is observed for the commercial positive control TSP-1 as a latent TGF-β1 activator. Thus, recombinant SARS-COV-2 NSP15 protein potently activates latent TGF-β1.

These novel data demonstrate that the SARS-COV-2 NSP15 protein has profound activation effects on human latent TGF-β1. The demonstrates further support the value of TGF-β1 inhibitors for the prevention and treatment of SARS-CoV2 related illness as disclosed and claimed herein.

### Example 3

The following experiment was performed to study the activity of existing commercially available pharmaceutical grade small molecule TGF-β1 receptor inhibitors: SB505124 and galunisertib (LY2157299). These inhibitors work via inhibiting the receptor TGF-β receptor 2 (TGFRβ2) which is required for canonical TGF-β1 signaling.

In these experiments, SB505124 and galunisertib (Fisher, Inc.) inhibitors were added at 1-2 uM concentrations to MLEC plated in 96-well plates using a 1 hour pre-incubation before addition of human active TGF-β1 in 0-320 nm concentrations with continued presence of the inhibitor for 18 hours overnight in standard conditions as described in Example 2 methods. A neutraceutical, the green tea catechin ECGC (Sigma) was also tested. Luciferase signals were determined after cell lysis and compared as in Example 2.

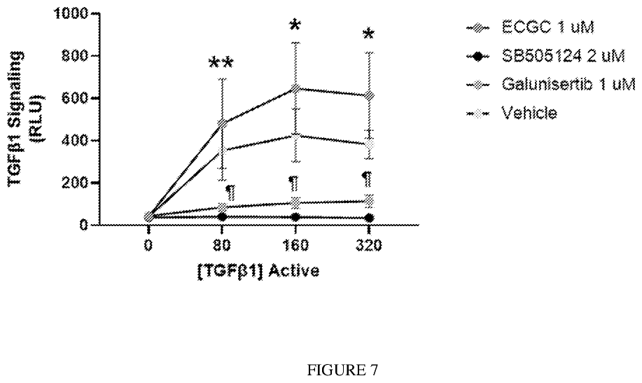

The data are shown in FIG. 7. These data show that SB505124 and galunisertib are potent at blocking active TGF-β1 signaling in mink lung epithelial cells at concentrations reported to be nontoxic in animal models and human cell lines. As explained above, mink lung cell line that is highly relevant to SARS-COV-2 infection since they express the ACE2 receptor. Mink are susceptible to infection with SARS-COV-2 (which necessitated the culling of millions of mink in Europe in early 2021 to suppress a large potential viral reservoir).

The TGF-βR2 inhibitors SB505124 and galunisertib (Fisher, Inc.) were effective at blocking more than 90% of active TGF-β1 signaling. In contrast, the green tea catechin ECGC was not effective at the 1-2 μm concentrations shown in FIG. 7 (and paradoxically augmented signaling at these doses), but displays 80 percent inhibition at higher concentrations in subsequent experiments (80 uM, not shown). It is noted that such concentrations in vivo may be associated with hepatotoxicity, a known side effect of catechins.

These results show that the TGF-βR2 inhibitors SB505124 and galunisertib (Fisher, Inc.) are potent inhibitors of active TGF-β1 in a mink lung cell line that is highly relevant to SARS-COV-2 infection.

### Example 4

The following experiment was performed to further study the activity of existing commercially available pharmaceutical grade small molecule TGF-β1 receptor inhibitors: SB505124 and galunisertib (LY2157299), along with an anti-TGFβ1 antibody. With these experiments, the appropriate concentrations of inhibitors were determined to assess their ability to block the effect of SARS-COV-2 NSP15 protein mediated activation of latent TGF-β1.

As in Example 3, MLEC cells were previously incubated for 1 hour with either PBS vehicle, 2 μg/ml of antibody, or 1-2 μM of galunisertib or SB505124 in this example. In this example, the SARS-COV-2 NSP15 recombinant protein produced in the inventor's lab was used in 0-2.4 nM concentrations to activate 2 nM latent human TGF-β1 in a 200 μL PBS reaction for 45 minutes at 37° C. in water bath. After incubations, 80 μL of each reaction mix (still containing NSP15 protein) was added to 320 μl of complete media, mixed, and added as 100 μl aliquots to individual wells of MLEC in triplicate, incubated overnight for 18 hours, then assessed for luciferase activity as described in the methods for the previous Examples.

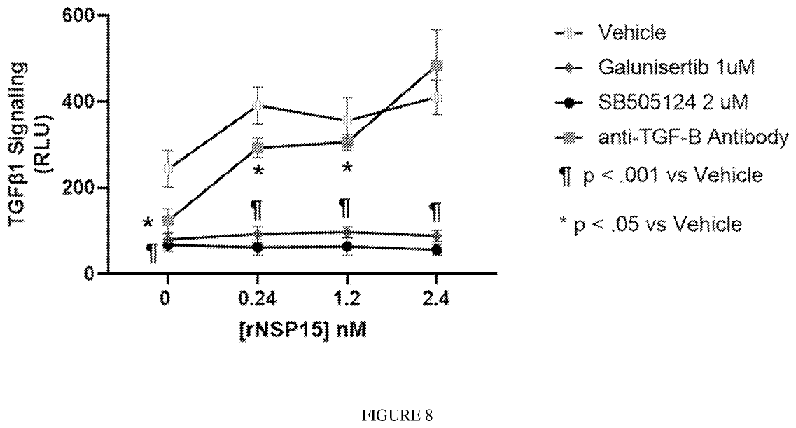

The data are shown in FIG. 8. The data indicate that TGFβ1 inhibitors block NSP15 effects on lung epithelial cell TGFβ1 signaling. Moreover, the data demonstrates that both small molecule TGF-β receptor 2 inhibitors as a class and an anti-TGF-β1 antibody as another drug class inhibit the downstream TGF-β1 signaling caused by NSP15 activation of human latent TGF-β1. As seen with experiments in prior Examples, the small molecule TGF-βR2 inhibitors were highly effective in blocking the effects of active TGF-β1—but this time as activated by the NSP15 protein. The antibody inhibitory effect was statistically significant and shows the potential benefit of another class of TGF-β1 inhibitors in SARS-COV-2 mediated illness, although it was less effective than the small molecule inhibitors in this experimental design. The difference in effectiveness is likely due to the fact that MLEC cells were preincubated for only 1 hour with antibody, and free TGF-β1 ligand from NSP15-latent TGF-β1 reactions would not have been fully bound without an overnight incubation of the reaction mix with antibody before addition to MLEC wells. One would expect a more robust inhibition of TGF-β1 signaling when similar antibodies are used in vivo.

The TGF-β1 inhibitors galunisertib, SB505124, and an anti-TGFβ1 antibody all block NSP15 mediated effects on TGF-β1 signaling in MLEC cells. The anti-TGFβ1 antibody was included as further proof of concept. Antibody therapy offers longer and pharmacologic inhibitory effects in humans and avoids potential small molecule TGFβR2-specific toxicities.

TABLE of
Sequences

| | | SEQ ID |
|---|---|---|
| Description | Sequence | NO. |
| SARS-CoV-2 | mhhhhhhssgvdlgtenlyfqsnamslenvafnvvnkghfdgqqgevpvsi | 1 |
| NSP15 | inntvytkvdgvdvelfenkttlpvnvafelwakrnikpvpevkilnnlgvdia | |
| | antviwdykrdapahistigvcsmtdiakkpteticapltvffdgrvdgqvdlfr | |
| | narngvlitegsvkglqpsvgpkqaslngvtligeavktqfnyykkvdgvvqql | |
| | petyftqsrnlqefkprsqmeidflelamdefieryklegyafehivygdfshsql | |
| | gglhlliglakrfkespfeledfipmdstvknyfitdaqtgsskcvcsvidlllddf | |
| | veiiksqdlsvvskvvkvtidyteisfmlwckdghvetfypklq | |
| KRFK peptide | KRFK | 2 |
| domain | | |
| WXXW | WXXW | 3 |
| (X = any amino | | |
| acid) | | |
| OT-101 | CGGCATGTCTATTTTGTA | 4 |
| (trabedersen; | | |
| AP12009) | | |
| synthetic peptide | LLIGLAKRFKESPFEL | 5 |
| centered on the | | |
| SARS-CoV2 | | |
| NSP15 KRFK | | |
| region | | |
| human KRFK- | DKRFKQDGGWSHWSPW | 6 |
| containing | | |
| peptide | | |
| thrombospondin-1 | | |
| (TSP1) | | |
| computer- | KFILALRKLLFESEGP | 7 |
| scrambled peptide | | |
| (SCR) | | |
| fragment from | TTLGGLHLLISQVRLSKMGILKAEE | 8 |
| HCoV-229E | | |
| fragment from | TTLGGLHLLISQFRLSKMGVLKADD | 9 |
| HCoV-NL63 | | |
| fragment from | KIIGGLHLLIGLYRRQQTSNLVVQE | 10 |
| HCoV-OC43 | | |
| fragment from | KVIGGLHLLIGLFRRLKKSNLLIQE | 11 |
| HCoV-HKU1 | | |
| KRFK motif | SQLGGLHLLIGLAKRFKESPFELED | 12 |
| fragment from | | |
| SARS-COV2 | | |
| NSP15 | | |
| fragment from | GQLGGLHLMIGLAKRSQDSPLKLED | 13 |
| SARS 2004 | | |
| fragment from | TTLGGLHLLIGLYKKQQEGHIIMEE | 14 |
| MERS-COV | | |

1. A method of preventing or treating a SARS-COV-2 infection in a subject in need thereof comprising administering to the subject a therapeutically effective amount of at least one transforming growth factor beta 1 (TGF-β1) inhibitor.

2. The method of claim 1, wherein the at least one TGF-β1 inhibitor blocks interaction of TGF-β1 and SARS-COV-2 NSP15.

3. The method of claim 1, wherein the at least one TGF-β1 inhibitor is selected from Galunisertib (LY2157299), LY580276, LY550410, SB505124, GS-1423, AVID200, Fresolimumab, LY2382770, LY3022859, and XOMA089.

4. The method of claim 1, wherein the at least one TGF-β1 inhibitor is or includes a selective TGF-β1 inhibitor.

5. The method of claim 1, wherein the at least one TGF-β1 inhibitor is targetable to lungs of the subject.

6. The method of claim 1, wherein the subject is at risk of infection by SARS-COV-2.

7. The method of claim 1, wherein the subject is infected by SARS-COV-2.

8. The method of claim 1, wherein the subject is administered two or more doses of the at least one TGF-β1 inhibitor.

9. A medicament for preventing or treating a SARS-CoV-2 infection in a subject in need thereof, comprising at least one transforming growth factor beta 1 (TGF-β1) inhibitors and one or more pharmaceutically acceptable carriers, vehicles, and/or excipients.

10. The medicament of claim 9, wherein the at least one TGF-β1 inhibitor is selected from Galunisertib (LY2157299), LY580276, LY550410, SB505124, GS-1423, A VID200, Fresolimumab, LY2382770, LY3022859, and XOMA089.

11. The medicament of claim 9, wherein the medicament is formulated for delivery to lungs of a subject.

12. At least one transforming growth factor beta 1 (TGF-β1) inhibitor for use in a method to prevent or treat a SARS-COV-2 infection in a subject in need thereof, the method comprising administering to the subject a therapeutically effective amount of the at least one TGF-β1 inhibitor.

13. The at least one TGF-β1 inhibitor of claim 12, wherein the at least one TGF-β1 inhibitor is selected from Galunisertib (LY2157299), LY580276, LY550410, SB505124, GS-1423, AVID200, Fresolimumab, LY2382770, LY3022859, and XOMA089.

14. The at least one TGF-β1 inhibitor of claim 12, wherein the at least one TGF-β1 inhibitor is or includes a selective TGF-β1 inhibitor.

15. The at least one TGF-β1 inhibitor of claim 12, wherein the method comprises administering the at least one TGF-β1 inhibitor to a subject at risk of infection by SARS-COV-2.

16. The at least one TGF-β1 inhibitor of claim 12, wherein the method comprises administering the at least one TGF-β1 inhibitor to a subject infected by SARS-COV-2.

17. The at least one TGF-β1 inhibitor of claim 12, wherein the method comprises administering two or more doses of the at least one TGF-β1 inhibitor to the subject.


SEQUENCE LISTING (14 sequences)

SEQ ID NO: 1 - protein, 371 aa, Severe acute respiratory syndrome coronavirus 2 (SARS-CoV-2)
  MISC_FEATURE (1)..(371): NSP15
MHHHHHHSSGVDLGTENLYFQSNAMSLENVAFNVVNKGHFDGQQGEVPVSIINNTVYTKVDGVDVELFENKTTLPVNVAFELWAKRNIKPVPEVKILNNLGVDIAANTVIWDYKRDAPAHISTIGVCSMTDIAKKPTETICAPLTVFFDGRVDGQVDLFRNARNGVLITEGSVKGLQPSVGPKQASLNGVTLIGEAVKTQFNYYKKVDGVVQQLPETYFTQSRNLQEFKPRSQMEIDFLELAMDEFIERYKLEGYAFEHIVYGDFSHSQLGGLHLLIGLAKRFKESPFELEDFIPMDSTVKNYFITDAQTGSSKCVCSVIDLLLDDFVEIIKSQDLSVVSKVVKVTIDYTEISFMLWCKDGHVETFYPKLQ

SEQ ID NO: 2 - protein, 4 aa, Artificial Sequence
  motif in NSP15
KRFK

SEQ ID NO: 3 - protein, 4 aa, Artificial Sequence
  ancillary motif peptide domain in NSP15
  MISC_FEATURE (2)..(3): any amino acid
WXXW

SEQ ID NO: 4 - DNA, 18 bp, Artificial Sequence
  synthetic antisense therapeutic
  misc_feature (1)..(18): OT-101 (trabedersen; AP12009)
cggcatgtctattttgta

SEQ ID NO: 5 - protein, 16 aa, Artificial Sequence
  synthetic peptide
  MISC_FEATURE (1)..(16): synthetic peptide centered on the SARS-CoV2 NSP15 KRFK region
LLIGLAKRFKESPFEL

SEQ ID NO: 6 - protein, 16 aa, Artificial Sequence
  fragment of thrombospondin-1
  MISC_FEATURE (1)..(16): human KRFK-containing peptide thrombospondin-1 (TSP1)
DKRFKQDGGWSHWSPW

SEQ ID NO: 7 - protein, 16 aa, Artificial Sequence
  synthetic peptide
  MISC_FEATURE (1)..(16): computer-scrambled peptide (SCR)
KFILALRKLLFESEGP

SEQ ID NO: 8 - protein, 25 aa, Artificial Sequence
  Fragment of accession no. KF514433
TTLGGLHLLISQVRLSKMGILKAEE

SEQ ID NO: 9 - protein, 25 aa, Artificial Sequence
  fragment of accession no. NC005831.2
TTLGGLHLLISQFRLSKMGVLKADD

SEQ ID NO: 10 - protein, 25 aa, Artificial Sequence
  fragment of accession no. AY391777.1
KIIGGLHLLIGLYRRQQTSNLVVQE

SEQ ID NO: 11 - protein, 25 aa, Artificial Sequence
  fragment of KF430201.1
KVIGGLHLLIGLFRRLKKSNLLIQE

SEQ ID NO: 12 - protein, 25 aa, Artificial Sequence
  fragment of NSP15 protein of accession no. NC045512.2
SQLGGLHLLIGLAKRFKESPFELED

SEQ ID NO: 13 - protein, 25 aa, Artificial Sequence
  fragment of accession no. AY502932
GQLGGLHLMIGLAKRSQDSPLKLED

SEQ ID NO: 14 - protein, 25 aa, Artificial Sequence
  fragment of accession no. NC019843
TTLGGLHLLIGLYKKQQEGHIIMEE